(19) United States
(12) Patent Application Publication
(10) Pub. No.: US 2008/0221396 A1
(43) Pub. Date: Sep. 11, 2008

(54) Method and System for Monitoring Medical Treatment

(75) Inventors: Maria Garces, Tampa, FL (US); Cathy A. Loffredo, Tucson, AZ (US); Michael Treat VanSickler, Columbia, MD (US); Christopher Brian Godschall, Ellicott City, MD (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Becton Dickinson and Company, Franklin Lakes, NJ (US)

(21) Appl. No.: 11/996,565

(22) PCT Filed: Jul. 24, 2006
(86) PCT No.: PCT/US06/28653

§ 371 Date: Jan. 23, 2008

Related U.S. Application Data
(60) Provisional application No. 60/702,319, filed on Jul. 25, 2005.

(51) Int. Cl. A61B 5/00; G08B 21/00

(52) U.S. Cl. 600/300; 340/870.16

(57) ABSTRACT

An electronic network is disclosed for effectively providing a technique to administer medical treatment. The technique pertains to at least one patient and at least one unit for medical treatment. The technique involves receiving patient data and unit data and evaluating at least the patient data and/or unit data to determine whether a match with preexisting data exists, which thereby generates an indication to the user to not proceed with the medical treatment in the absence of such a match.

### CROSS REFERENCE TO RELATED APPLICATIONS

This application claims the benefit of the filing date of U.S. Provisional Application No. 60/702,319, filed Jul. 25, 2005, the disclosure of which is hereby incorporated by reference.

### FIELD OF THE INVENTION

The present invention relates to an electronic network for monitoring patient-specific or time-sensitive treatments and, more specifically, to an electronic network for monitoring and reducing errors that may occur in blood transfusions.

### BACKGROUND OF THE INVENTION

Laboratory Information Systems (LIS) and Hospital Information Systems (HISs) both fall under the category of Health Care Information or Enterprise Systems. Generally, healthcare enterprises provide various aspects of patient care such as patient identification and tracking, as well as medication, sample collection, sample order and data management. In providing patient care, healthcare workers typically utilize one or more software applications accessible through a healthcare information system to access the most current information about a patient and the status of their care.

Access to healthcare information systems have typically, in the past, required fixed terminals such as nurse workstations to be used at a location potentially distant from the point of care/delivery of medical services (i.e., at the patient's location). To provide more convenient and efficient access to an LIS, more portable modules such as handheld computers or portable data terminals (PDTs) have recently been introduced into healthcare and hospital settings and are hereinafter generally referred to as “handhelds” or “handheld devices”. The handhelds can be connected to a server directly through a LAN, modem, wireless connection, or the like. Optionally, the handhelds can be connected to a server through a PC using a serial or parallel connection. In order to use the handheld, the information on the handheld is synchronized with the LIS by connecting the handheld to a data import/export device connected with the LIS, or via a cable connected with the LIS, to allow the exchange of data between the LIS and the handheld.

Although portable communication systems have been deployed to provide accurate, real-time, comprehensive information about a patient to medical personnel at any location, there are still many points in the delivery of medical services where real-time or recently updated information is needed to inform decisions in providing care. Clearly, almost any point in the delivery of medical services could benefit from being able to receive such information via a portable terminal. However, each point in the delivery of such services is unique in terms of the information that is required, how that information is presented (i.e. displayed) in support of the decision that is to be made with regard to patient care. Accordingly, systems that ensure the integrity of the provided medical services continue to be sought.

### SUMMARY OF THE INVENTION

In modem medicine, few treatments are generic in the sense that they are interchangeable from patient to patient. Even the simplest medications are customized by composition, dose, etc. for the patient that is to receive it. Furthermore, many treatments are time-sensitive in that they must be administered within a certain period of time in order to ensure that the treatment is effective. Still further, medicines and other products used for treatment often arrive through a complicated logistical path involving multiple people and multiple locations.

For example, managing blood and blood products used for transfusion is an extremely complicated problem. Examples of “blood” and “blood products” (used interchangeably hereinafter) include, but are not limited to whole blood, packed cells, packed cells irradiated, packed cells leuko reduced, cryoprecipitate, platelets, and/or fresh frozen plasma. Blood transfusions must be carefully monitored for a variety of reasons.

In order to better administer aspects of blood transfusions, it is useful to track units of blood (hereinafter “blood units” or “units”) and patient identification information. Improperly administered transfusion of blood and other fluids into a patient may cause complications or lead to a fatality. Hospitals and other medical institutions are making increased efforts to design better transfusion systems to reduce the risks associated with blood transfusions such as transfusing a patient with an incorrect blood type or outdated blood and/or blood components. Elements of a transfusion system that may be subject to error include, but are not limited to, incorrect collection of samples, incorrect handling of blood and/or blood components, incorrect labeling of blood and incorrect ordering of blood for a patient.

Aspects of the invention monitor the medical treatments, such as blood transfusion, to ensure that the appropriate unit(s) is used in the process and to ensure that the patient is receiving the treatment using effective practices.

In one embodiment of the present invention a system and method for monitoring a unit, such as a blood unit, includes receiving data indicating an amount of time that the unit is released from, for example, a blood bank and then comparing whether the amount of time exceeds a predetermined time threshold. A message may be generated when the amount of time exceeds the predetermined time threshold. In addition, when the amount of time is exceeded, the display message indicates that the unit is to be returned to unit originator, such as the blood bank, or that the unit is to be disposed.

In another embodiment of the invention, data is received indicating a duration that the unit is released and indicating a duration that the unit has been previously released. The durations are totaled and a comparison is made as to whether the totaled amount of time exceeds a predetermined threshold. A message is generated when the totaled amount of time exceeds the predetermined time threshold. In one embodiment, the display message indicates that the unit is to be disposed.

In a further embodiment of the invention, unit data is received upon a request for a unit and determination is made, based upon the unit data, as to whether the unit has been designated for disposal or was previously requested. A message is generated when the unit has been designated for disposal or was previously requested.

In yet another embodiment of the invention, a medical treatment start time and medical treatment end time are received and a determination is made, based upon the medical treatment start time and the medical treatment end time, as to whether the medical treatment has exceeded a predetermined time threshold. A message is generated when the medical treatment has exceeded the predetermined time threshold.

In a further embodiment of the invention, information is received relating to the location of the medical treatment as well as a start and end time of the medical treatment. A sensor is configured to determine whether a device associated with the administrator of the medical treatment is in the location of the medical treatment during the start and the end time. A message is then generated when the device associated with the administrator of the medical treatment is not in the location of the medical treatment during the start and the end time.

In yet a further embodiment of the invention, start time and end time of the medical treatment is received. A request is then generated instructing the administrator of the medical treatment to scan identification information associated with a label of the patient between the start time and the end time. A message is generated if no identification information associated with a label of the patient between the start time and the end time is received.

In yet another embodiment of the invention, patient-specific unit source information and information pertaining to a source of the medical treatment unit to be provided to a patient are received. A determine is made as to whether the patient-specific medical treatment unit source information matches the source of the medical treatment unit to be provided to the patient A message is then generated when the patient-specific medical treatment unit source information does not match the source of the medical treatment unit to be provided to the patient. In one aspect of the invention, the processor requests emergency information, when the patient-specific medical treatment unit source information does not match the source of the medical treatment unit to be provided to the patient. Unit source information may relate to the following categories of units, as described below in the detailed description: allogenic (from a random donor), self-directed (from a particular donor) and autologous (from the patient).

### BRIEF DESCRIPTION OF THE DRAWINGS

These and other aspects, advantages and novel features of the present invention will be readily comprehended from the following detailed description when read in conjunction with the accompanying drawings.

FIGS. 1 through 4 each illustrate a client handheld and server configuration that perform various processes, in accordance with different embodiments of the present invention;

FIGS. 5 through 17 depict respective client handhelds with exemplary display screens, in accordance with an embodiment of the invention;

FIG. 18 illustrates a flowchart that depicts the process for receiving unit status information, in accordance with an embodiment of the invention;

FIG. 19 illustrates a flowchart that depicts the process for handling a unit request, in accordance with an embodiment of the invention;

FIG. 20 illustrates a flowchart that depicts the process for monitoring the time for allowing a unit to be returned to a blood bank, in accordance with an embodiment of the invention;

FIG. 21 illustrates a flowchart that depicts the process for monitoring the total time that a unit is released from a blood bank, in accordance with an embodiment of the invention;

FIG. 22 illustrates a flowchart that depicts the process for monitoring the disposal of a unit, in accordance with an embodiment of the invention;

FIG. 23 illustrates a flowchart that depicts the process for monitoring time between commencement and termination of a blood transfusion, in accordance with an embodiment of the invention;

FIG. 24 illustrates a flowchart that depicts the process for monitoring presence of healthcare personnel during transfusion process, in accordance with an embodiment of the invention; and

FIG. 25 illustrates a flowchart that depicts the process for monitoring whether appropriate unit category is used for transfusion, in accordance with an embodiment of the invention.

### DETAILED DESCRIPTION OF THE PREFERRED EMBODIMENTS

### System

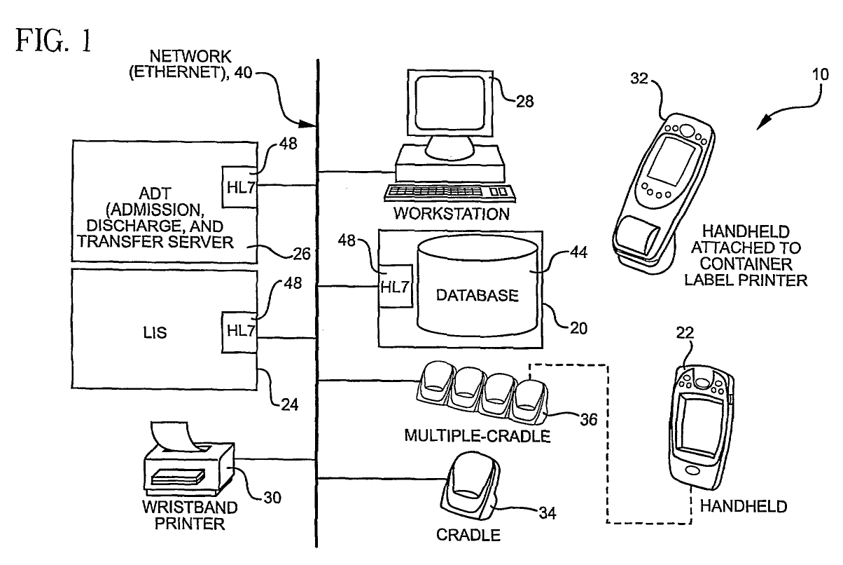

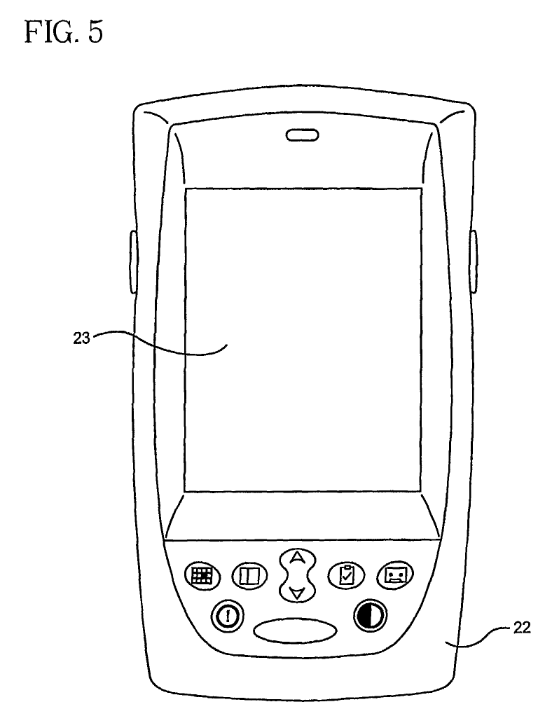

In accordance with an embodiment of the present invention, a blood transfusion monitoring and management system 10 (FIGS. 1-4) and portable (i.e. handheld) medical device 22 (FIGS. 5-17) are provided. The backbone of system 10 is described generally in WIPO International Publication Number WO 2005/111086A2, which was filed on Apr. 28, 2005, and which is commonly assigned and is entitled “System and Method for Medical Error Monitoring” (claiming priority U.S. Provisional Application No. 60/575,244, filed on May 28, 2004; U.S. Provisional Application No. 60/571,434 filed May 14, 2005 and U.S. Provisional Application 60/566,439 filed on Apr. 30, 2004 under 35 U.S.C. 119(e)), and is incorporated by reference herein. FIGS. 1-4 illustrate variations in systems 10 in which the network 40 links with different information systems (e.g. LIS 24, 11S 38, ADT 26). The network can take the form of a cable-based or fiber optic network, a local are network (LAN), or wide area network (WAN) a virtual private network (VPN), the Internet, or any other type of network that allows communication between computing devices.

In one embodiment, the handheld device 22 incorporates a scanner for scanning labels that contain unit information, patient information or both. Examples of such labels include patient identification labels, unit labels, hospital order labels, Hollister labels etc.

The scanner may scan a one-dimensional bar code, two-dimensional bar code, or any other machine readable code. For illustrative purposes, the system 10, including handheld device 22, will be described herein with reference to blood transfusion procedures. It is to be understood that the system 10, including handheld devices 22, can also be used for monitoring the administration of other agents to patients.

In accordance with an embodiment of the invention, the handheld device or PDT 22 (FIG. 5) allows a user (the term “user” is used herein to describe a person who uses a handheld device to track samples of specimens collected from patients, especially in the healthcare setting) such as a nurse, doctor or phlebotomist to access information regarding the units and, among other things, to match such information with the patient information (e.g., scanned from the patient wristband) and confirm that the unit (not shown) is the correct one for the patient. When the handheld device 22 is replaced in its cradle 34 (see FIG. 1), the handheld synchronizes with the specimen management server (SMS) 44, which is then able to communicate with the LIS 24. In the embodiments described herein, SMS contains unit data and has the ability to query other systems on the network for unit data or patient data and to store that data. Consequently, SMS server is also referred to herein as blood transfusion server (BTS) or simply server.

With reference to FIGS. 1 through 4, the system 10 preferably comprises a server 20 (e.g., containing unit data in database 44), a plurality of client handhelds 22 with data accessibility to the SMS 20, a LIS (Laboratory Information System) 24, and an ADT (Admission, Discharge and Transfer system) 26. The system components are connected to a network 40 to allow for specific communication events to occur. Other embodiments might include aspects of the server 20 embedded into other systems (e.g. the LIS 24). The handheld device 22 communicates with hospital computer systems (e.g., the LIS, ADT and HIS) via the server 20 and network 40. In alternate embodiments, the system 10 is configured for direct communication between the handheld and other systems (the LIS 24, for example).

To better understand the present invention, certain terms shall be defined as follows:

### Client

The client is the handheld device 22 that can download files and data for manipulation, run applications, or request application-based services from a file server.

### Cradle

A cradle 34 is a docking station used to provide an interface with the system 10. The cradle 34 can be adapted to receive and secure the handheld device 22. A detector element can be included to detect when the handheld device 22 is placed in the cradle. Data can be received from a server 20 and selectively downloaded when the handheld 22 is placed in the cradle. In the embodiments described herein, the server 20 may comprise a BTS. An actuator on the handheld device 22 can be employed for initiating the transfer of data to a process in the host terminal if the detector indicates that the handheld device 22 has been placed in the cradle 34.

### Database

The term database includes one or more large structured sets of persistent data, such as database 44, usually associated with software to update, insert, and query the data.

### Handheld Device

The term handheld (e.g., handheld device 22) describes portable computers useful for blood transfusion management at the point of use. One example of such a portable handheld element is the Symbol Technologies PPT 1700 Series handheld. This handheld device has infrared (IR) and barcode scanning capabilities. The handheld comprises a graphical user interface (GUI) for displaying information useful during blood transfusion processes. However, the scanning feature of the handheld is optional.

The handheld device 22 typically includes a processor. In the system of an embodiment of the present invention, the processor can be located in the handheld device 22 or in another part of the system 10 (e.g. the BTS 20). The processor is configured to process data relating to the patient identification information or unit information (e.g. Hollister code).

As previously noted, the handheld device 22 is optionally equipped with a scanner or other type of reader. In the described embodiments of the present invention, the data is entered into the handheld device 22 directly by the operator, or it is otherwise received by the scanner or reader. For example, a barcode scanner reads identification information from a patient identification code or Hollister code printed on a patient's wristband.

In an embodiment of the present invention, the scanner is a miniature code reader. The code reader could be a barcode scanner, imager, infrared identification reader, RFID reader or similar technology. The barcode scanner can either be integrated into the handheld device 22 or attached to the handheld device 22 via an accessory device.

Likewise, an RFID reader may be integrated into the handheld device 22 so that when in proximity to the agent (e.g., blood) container, the information transmitted by the container's RFID device, if available, could be read by the reader.

The handheld device 22 can optionally include a variety of other features and accessories. The features enumerated herein are by way of illustration, and not by limitation. For example, the handheld device 22 may also include a battery, a display screen for the graphical user interface (GUI), depressible keys, communication circuitry, a memory element, housing for securing all the handheld subcomponents and a speaker for emitting an audible sound.

In another embodiment, a handheld device 22 can be secured to a sled accessory (not shown) that contains a barcode scanner. The sled accessory can have latching mechanisms that allows the user to irremovably secure the sled accessory to the handheld device 22. When the two components are so engaged, a communications port on the sled accessory is in communication with a similar port on the back of handheld device 22. This connection allows the transfer of data and information between the two components.

The handheld device 22 can be configured in a number of different ways, as will be appreciated by one skilled in the art. For example, the handheld device can be configured as a portable digital assistant (PDA), tablet PC, or notebook computer that includes a module and/or software for communicating with a server.

In an embodiment, an information code reader (not shown), such as a barcode reader, is used to input data into the system via handheld device 22. The barcode reader can be attached to the handheld device or can be a separately held wand that transmits signals to the handheld device. Input data received through the barcode reader includes, but is not limited to, codes or other indicia placed on units, patient ID tags, Hollister coded wristbands, and health practitioner personnel tags. For example, the system 10 and handheld device 22 are configured such that a user can remove the handheld device 22 from its cradle and then scan his or her ID badge to communicate to the handheld device 22 who is currently using the handheld device 22 and who will be performing the transfusion/collection of blood units within a given hospital ward, section, or floor.

### HIS

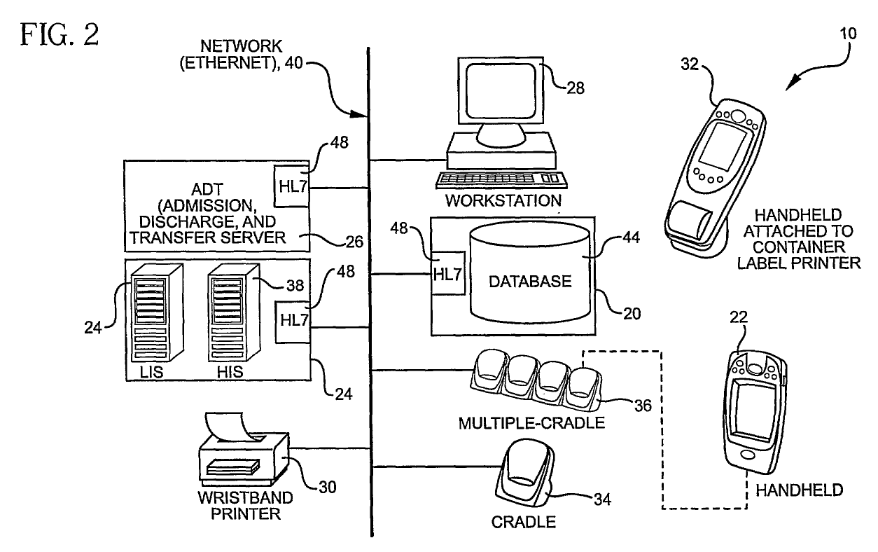

The Hospital Information System (HIS) 38 (FIG. 2) is a system developed with the objective of managing and streamlining the treatment flow of a patient in the hospital, along, with all data associated with the patient necessary for efficient and organized healthcare service. The HIS 38 allows doctors and other staff to perform to their peak ability in an optimized and efficient manner. Most HISs are modular, thus ensuring sustained benefits through changes in technology such as integration with new and improved LIS systems 24 (described below). Treatment flow can include, but is not limited to, blood transfusion management. As described more fully below, blood transfusion management include information about the patient's blood type as well as information about the blood that the patient is designated to receive (i.e., the patient's own blood, blood designated for the patient or a transfusion from the general supply bank).

HISs 38 use a network of computers to gather, process, and retrieve patient care and, administrative information for most hospital activities. HISs 38 can be configured in a variety of ways to satisfy the functional requirement of the users. HISs 38 also provide decision support systems for hospital authorities developing and managing comprehensive healthcare policies.

HISs 38 incorporate integrated computerized clinical information systems for improved hospital administration and patient healthcare. They also provide for accurate, electronically stored medical records for one or many patients. Typically, HISs 38 are centralized information systems designed for quick delivery of operational and administrative information and include software capable of optimizing core data and other application modules that can be customized to the hospital or healthcare facility.

### LIS

The term LIS 24 preferably defines a computer network comprised of industry standard network hardware and software (network and communication protocols) that serves to allow communication between the patient health record repository, the end-user client applications running on various device types, and the various types of servers. This network can take the form of a cable-based or fiber optic network, a local area network (LAN), a wide area network (WAN), a virtual private network (VPN), the Internet, or any other type of network that allows communication between computing devices.

The LIS 24 typically organizes and tracks information pertaining to laboratory tasks such as how orders are generated and communicated to the lab, how patients or units are delivered, how the units are accessioned and prepared, how testing is actually accomplished, and how results are communicated to healthcare providers. LISs can also organize, track, and determine how the health enterprise is reimbursed for the work done in the lab, and how the reimbursement, information is exchanged.

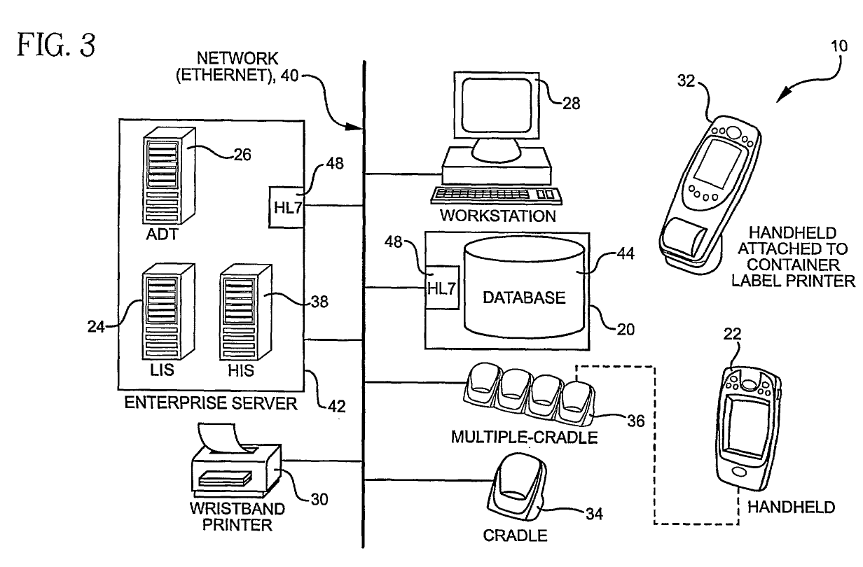

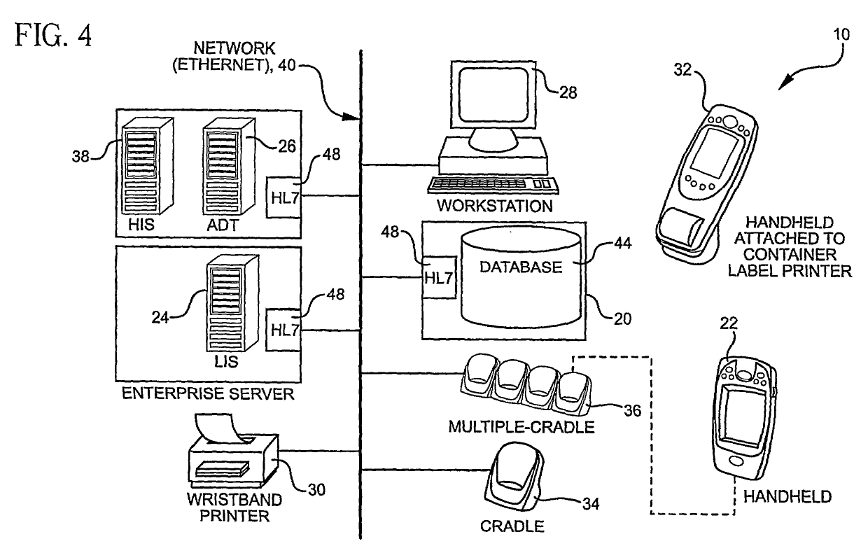

As shown in FIG. 2, an enterprise server 42 can include the LIS 24 and the HIS 38, or the Admission, Discharge and Transfer System (ADT) 26, LIS 24 and the HIS 38, as shown in FIG. 3. Alternatively, the HIS 38 and the ADT 26 operations can be combined in a single server (FIG. 4), among other configurations. In one embodiment described herein, the LIS 24 has a bi-directional interface with the server 10. Through this interface, blood transfusion inventory information sent from the LIS to the server 20, and data and canceled orders are sent from the server 20 to the LIS 24.

### LIS/HIS Data Interface

The LIS/HIS data interface 48 is an element that allows for facilitated communication for multiple modules sending and receiving data packets and signals across a network. Examples include Health Level Seven (i.e. HL7 3.0), ASTM 1238, ASTM 1394, Dbase, Comma Delimited ASCII, and Fixed Length ASCII. It is through this interface that the various elements of the system communicate with each other.

In one embodiment of the present invention, the system has a webpage that presents information regarding blood transfusions from the server. The webpage may be updated automatically based on a time interval set by a user. Referring to FIG. 1, the webpage is displayed on computer terminal 28

### Graphical User Interfaces

Referring to FIGS. 5 through 17, different handheld device 22 screen displays or GUIs are shown for the blood transfusion monitoring system according to one embodiment of the present invention.

The handheld device comprises a display 23, such as a liquid crystal display. This display is used to communicate information from the system to the user. In certain embodiments, the display 23 may be touch-sensitive. Using the touch sensitive feature, a user can use their fingers or a touch pen to enter information into the system 10.

The system 10 also includes a memory for recording orders and events associated with the blood transfusion/agent administration process. The memory is, for example, an internal fixed memory on a semiconductor integrated circuit (IC). Alternatively, the memory may be removable semiconductor memory such as flash drive, MemoryStick®, SD card, XD card, or any other commercially available external memory. In one embodiment, this memory is located in the handheld device 22.

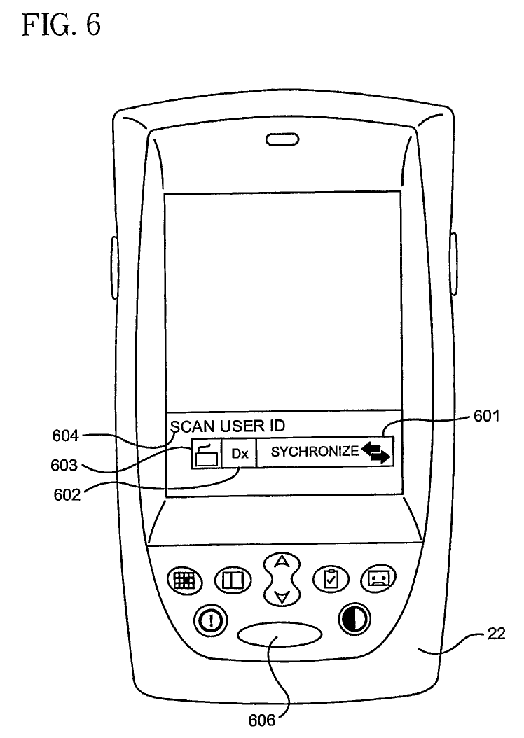

FIG. 6 illustrates the handheld device 22 display screen when the blood transfusion process begins. The screen displays a synchronize icon 601. When activating (e.g. by touch) synchronize icon 601, synchronization of the handheld device 22 with the server 20 may take place, via, for example, a wireless connection or through the cradle 34 (FIG. 1) if placed therein. Icon 602 is used to switch access between a transfusion management system (Tx) and a specimen management system or database (Dx). If the handheld device 22 is set for Tx, then icon 602 displays Dx. Alternatively, if the handheld device is accessing the database, icon 602 displays Tx. Keyboard icon 603 allows a user to access a keyboard graphic allowing a user to type in information, enter passwords, etc. when the icon is activated.

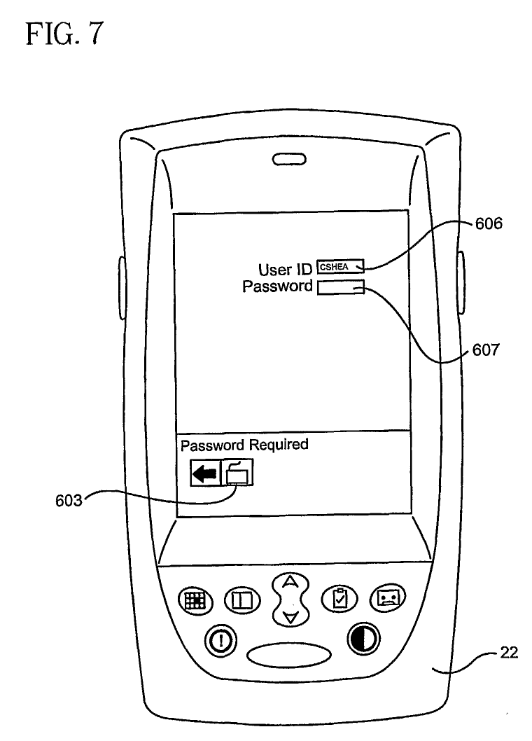

As shown in FIG. 6, the handheld device 22 prompts the user to enter (e.g., scan) in his/her user ID by prompt 604. Scan button 606 is used to activate the scan feature of the handheld device 22. After the user ID is scanned, the handheld device 22 may prompt the user to enter a password 607 associated with the user ID 606 as shown in FIG. 7. In the illustrated embodiment, the keypad 603 will be touched or otherwise activated for the user to enter their password. If the password does not match, the handheld device 22 will display an error message and/or issue an audible sound indicating the error. This prevents unauthorized users from using the handheld device 22 and performing blood transfusions using system 10.

Additionally, different users may have different access rights/permissions. These rights/permissions include, but are not limited to, viewing transfusion tracking data, editing transfusion tracking data, generating transfusion management reports, enabling pre-transfusion signoff, receipt of units issued, return of units to the blood bank, transfusion of product, etc.

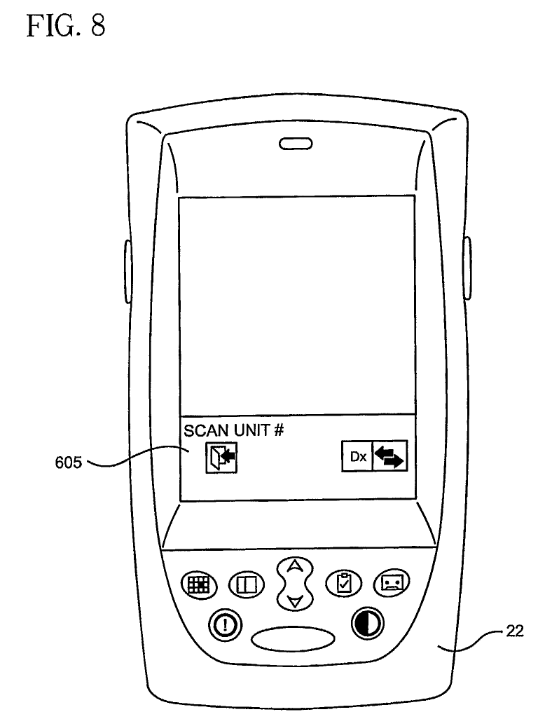

After the user has been authenticated, the handheld device 22 prompts the user to scan the unit number associated with a blood unit, as shown by prompt 605 in FIG. 8. When the unit information is received by for example server 20, database 44 is accessed to obtain the information relevant to the sample. For information to be accessed, the scanned unit information must match the sample identifier in the data base for that unit.

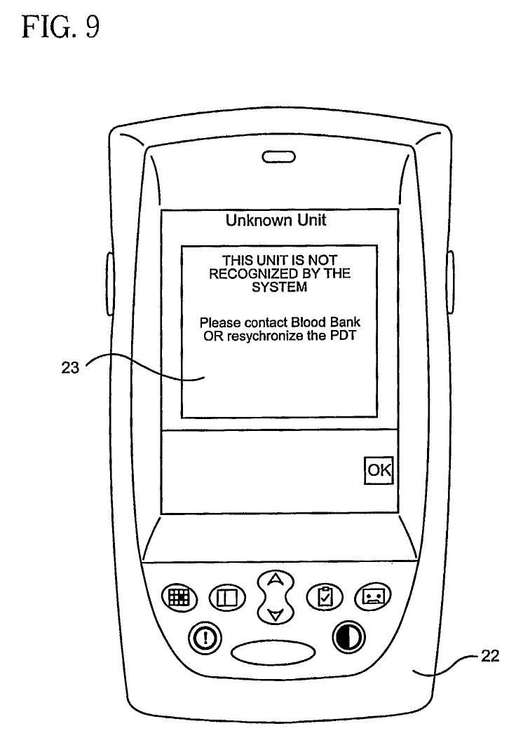

FIG. 9 illustrates the display on the handheld device 22 if the unit number is not recognized by the system 10. In such an event, the screen 23 displays an error message that indicates the unit is not recognized. This may happen for various reasons including, but not limited to, the unit being misdelivered or the handheld device 22 not being properly synchronized before the unit number is scanned.

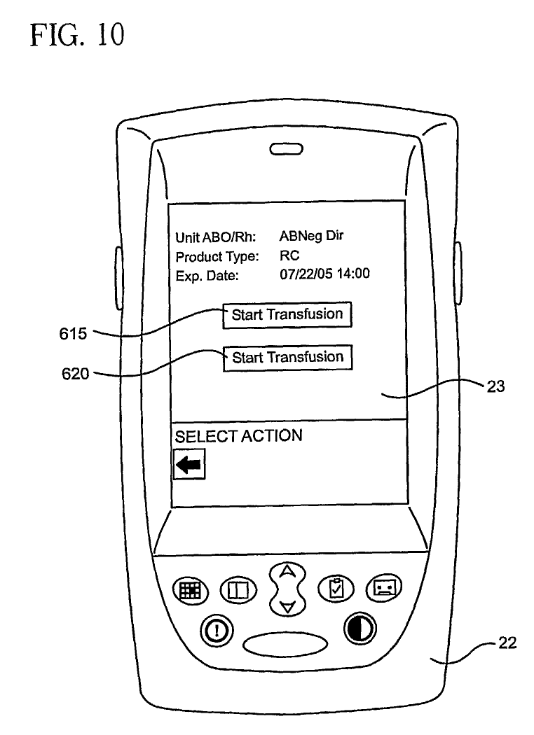

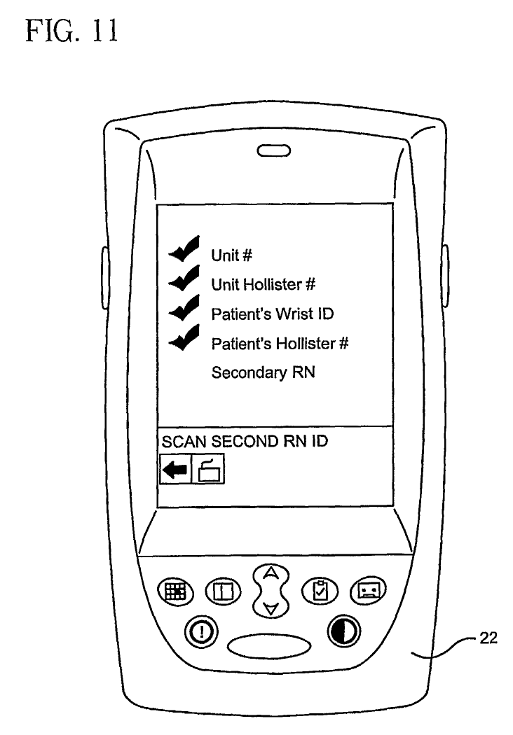

FIG. 10 shows a display screen from which a user can control/manage the transfusion process. The screen 23 has two actions that the user can select: 1) start the transfusion (615); and 2) stop the transfusion (620). If the user selects “Start Transfusion,” the handheld device will display a checklist as shown in FIG. 11. The user selects between starting and stopping the transfusion by touching the desired action on the display 23. The checklist of FIG. 11 prompts the user to perform a number of actions/obtain certain information. For example, the handheld device prompts the user to scan the unit number and then the unit's Hollister number. The Hollister number is a secondary form of identification issued by a blood bank. The Hollister number may be color coded and be a pre-assigned number depending on the type of product. As indicated in FIG. 11, once the requested information is provided, a check mark appears adjacent the field that indicates that the requested information has been received. The screen 23 also displays information about the blood sample at this point (e.g. blood type, expiration date, etc.).

Due to the high risk involved in blood transfusions, many hospitals and other medical institutions may require a second person to be present during blood transfusions. In this embodiment, the handheld device 22 is configured to prompt the user to scan the ID of a secondary registered nurse (RN). After the secondary RN's ID is scanned, the secondary RN is prompted to provide a password on the password screen as shown in FIG. 7.

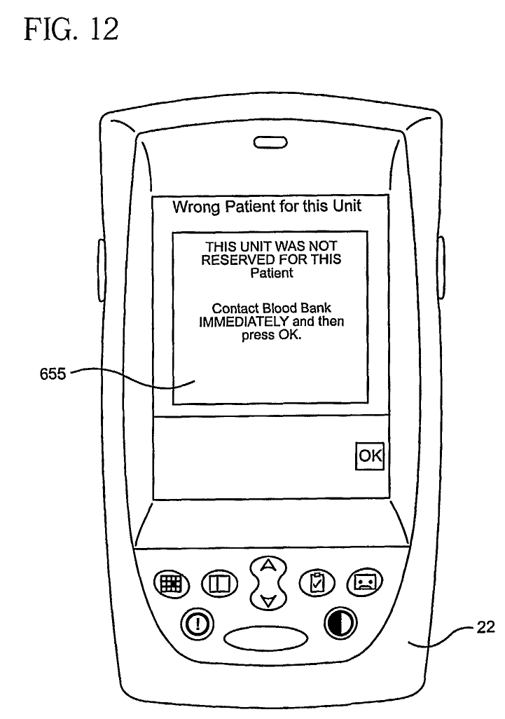

If the unit information stored in database 44 and/or handheld device 22 yields that the unit is not reserved for or compatible with the patient, as described below, an error message, such as message 655 of FIG. 12 is displayed.

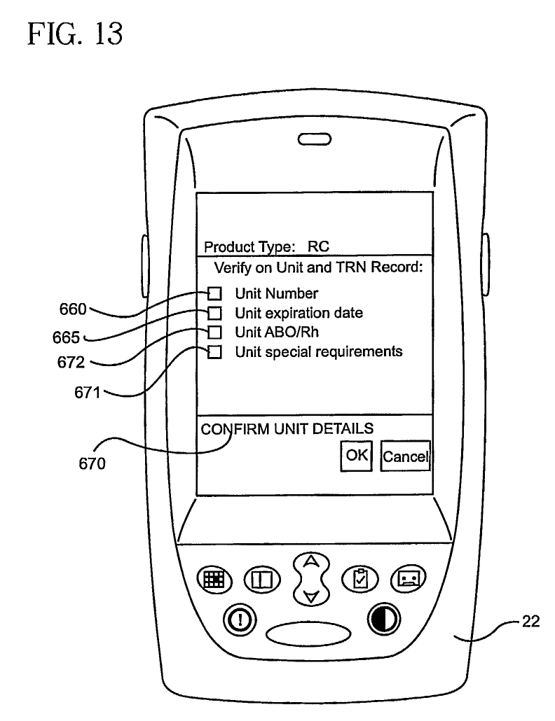

FIG. 13 illustrates another checklist that may be displayed on the handheld device. In FIG. 13, the handheld device prompts the user to confirm details 670 about the scanned unit to ensure correspondence between the unit and the transfusion record. These may include the unit's number 660 such as an accession number and the unit's expiration date 665.

Units typically have a limited shelf life and therefore have an expiration date. The expiration date depends on the type of blood product, the conditions under which the blood products were stored, etc. For example, a packed cell blood product may have an expiration date of six to seven weeks after it has been extracted from a donor. Once it is removed from a blood bank, there is a limited time period before the unit must be used, returned to the blood bank or discarded. If it is not going to be used, a user may have for example thirty minutes to return the unit before it is no longer viable. System 10 may be configured to automatically monitor the expiration time/date and alert the user that the unit has expired via a visual indication or an audible indication on handheld device 22. A color coded system may also be used to provide the alert, wherein when the unit is released for 0 to 10 minutes a green display is provided, when the unit is released between 10 and 20 minutes a yellow display is provided and when the unit is released between 20 to 30 minutes a red display is provided.

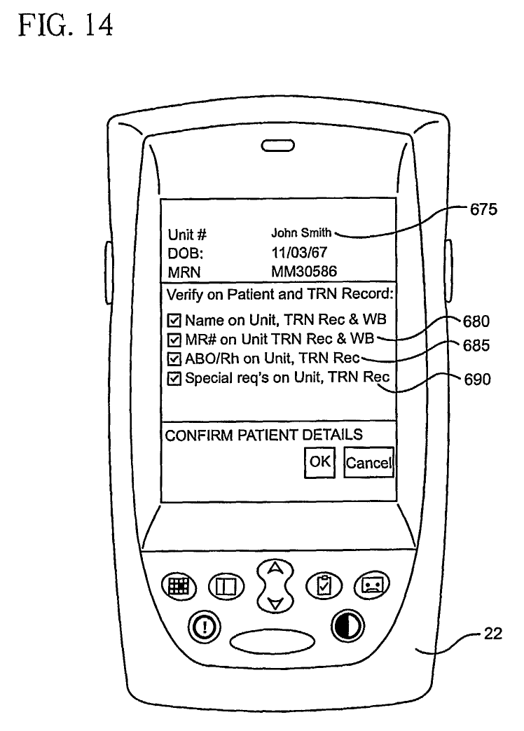

FIG. 14 depicts another checklist that may be displayed on the handheld device 22. In FIG. 14, patient data is confirmed as it is reflected in the transfusion record. The fields illustrated in FIG. 14 may include, e.g., the name of the patient 675, the medical record number on the unit 680 and blood type on the unit 685 are confirmed to ensure that the unit is given to the correct patient. The screen in FIG. 14 also includes a field 690 for special requirements on the unit and the transaction record.

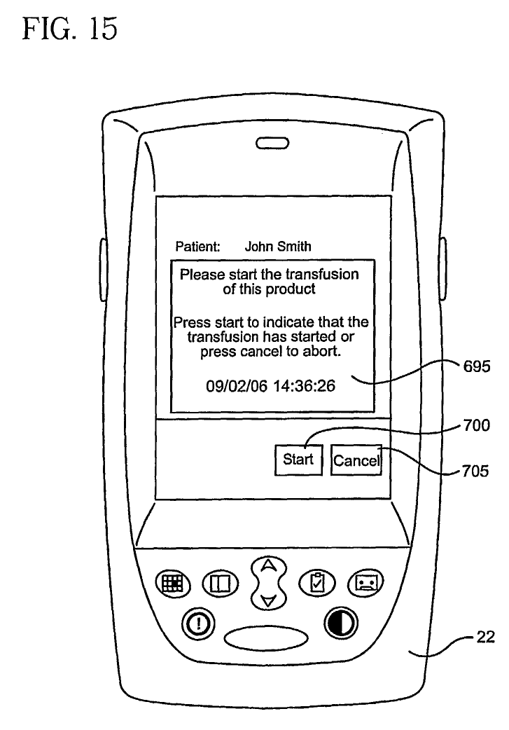

Referring to FIG. 15, after the unit and patient details are confirmed, the user may be prompted by message 695 to start the transfusion. The user may then elect to start the transfusion process by selecting start icon 700 or to cancel the transfusion process by selecting cancel icon 705. If the user selects start, this provides an input to the handheld device 22 that causes the system to keep a record of the date and time that the transfusion is started as shown in 695 of FIG. 15.

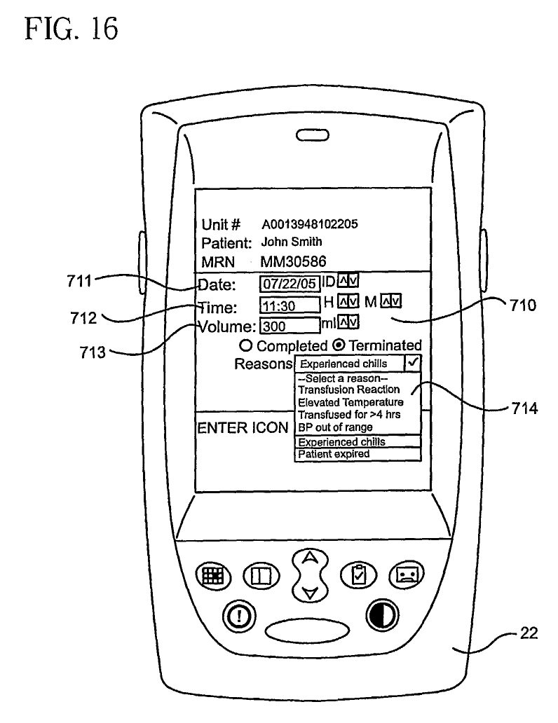

Once the transfusion has commenced, the handheld device 22 may be used to receive and transmit history data regarding the transfusion. This information is entered into the system 10 using the screen illustrated in FIG. 16. FIG. 16 displays a termination screen 710 that appears when the user presses the stop transfusion icon in FIG. 10. When the termination screen is displayed, the user enters a date 711 and time 712 that the transfusion was terminated. In other embodiments, the date and time fields are filled in automatically when the transfusion icon is activated. The volume that was transfused 713 may also be entered.

In addition, the reason the transfusion is terminated 714 may be entered. In this embodiment, illustrated in FIG. 16, the handheld device 22 provides a dropdown menu from which a reason for termination is selected. The transfusion may have been completed or it may have been terminated for other reasons which may include, but are not limited to the following:

transfusion reaction

patient expired

unit issued for longer than authorized duration

elevated temperature

blood pressure out of range

pain

chills

hives

shock

hematuria

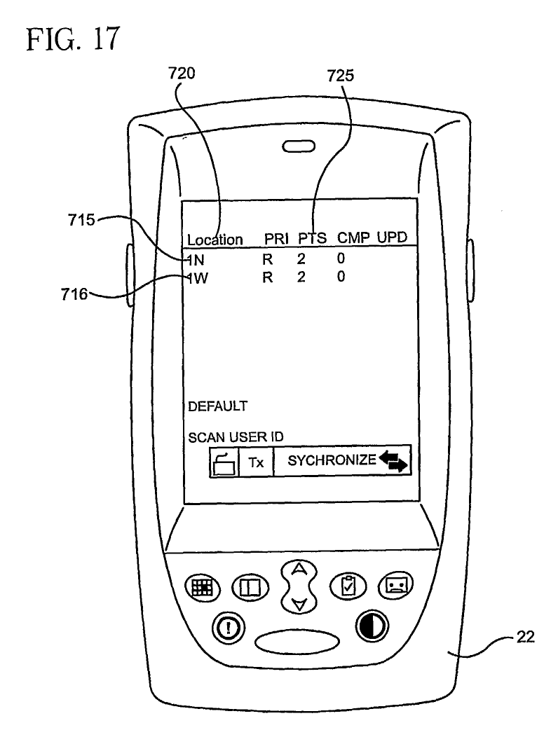

FIG. 17 depicts a handheld device 22 when it is accessing unit data. The handheld device 22 can track different units by displaying unit records, for example unit records 715 and 716, the location 720 of the units, and the patient 725 for which the units are intended. The unit records may also be color coded to indicate the amount of time that has elapsed since the unit was issued from the blood bank.

Likewise using workstation 28, a user may access system 10 to view and/or edit transfusion data (providing, in one embodiment, the user has the appropriate authorization). In addition, workstation 28 may receive (and in some cases process) information regarding one, some or all blood units of a given setting. The transfusion data may be separated into various categories, such as units that have been issued and units that are being transfused. Further, each unit may be associated with various type of information, including, but not limited to, a patient (name or other identification data), the patient's location in a hospital or some other healthcare setting, identification information regarding the issued unit, the type of unit, when it was issued, when it was received, and the time remaining to return the unit before the unit is no longer useful.

Thus, as illustrated in the GUIs of FIGS. 5-17, handheld device 22 may be used to receive information and display information for managing and administration of certain aspects of the transfusion process—such as tracking units that have been requested for release from the blood bank.

### Monitoring Processes

As described above, the handheld device 22 is equipped with a display screen, which allows for communication between a healthcare provider's central system, such as BTS 20, and healthcare provider's users, through for example handheld device 22. Such a network enables various processes, as described below, for effectively conducting blood transfusions.

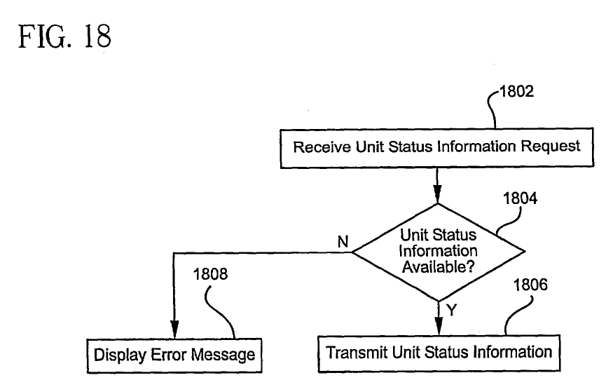

For example, turning to FIG. 18, the status of one or more units may be conveyed to a user that performs a unit request. Thus, if a user, through for example handheld device 22, makes a request for unit status information (step 1802), in accordance with an embodiment of the invention, handheld device 22 determines whether status information is available (step 1804). If unit status information is available, such information may be sent to handheld device 22 (either automatically or upon a user requesting such information) (step 1806). If, however, such information is not found, then an error message is displayed (step 1808). Unit status information may comprise various types of unit data, including the type of blood contained in the unit, the unit location, whether it has been allocated to a patient, and the like. The absence of unit status information may arise for various reasons, including the unit identification information is incorrect, handheld device 22 (and/or database 44) does not have the most up to date information, etc.

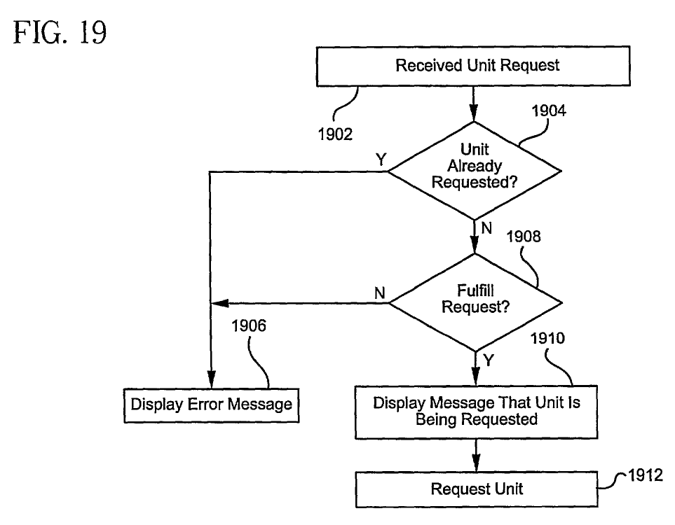

In addition, as illustrated in FIG. 19, system 10 may also determine whether the unit has already been requested by a user. At step 1902, handheld device 22 receives a unit request. The device 22 then determines whether the unit has already been requested (step 1904). If the unit has already been requested, then an error message is displayed indicating that a previous request has been made (step 1906). If, however, the request has not been previously made, then handheld device determines whether to fulfill the request based upon one or more protocols provided in detail below (e.g., in connection with FIG. 13), that evaluates whether a match between the patient and the unit exists (step 1908). If the request is not to be fulfilled (e.g., there is no match), an error message is displayed (step 1906). If, however, handheld 22 determines that request is to be fulfilled, then a message is displayed informing the user that the unit is being requested (step 1910) and the unit request is fulfilled (step 1912).

System 10 may also track the time that has elapsed since the blood bank issued the unit. Specifically, the time that the blood bank issued the unit is tracked and the time elapsed since the unit issued is computed by the processor (which may be located in the handheld device 22 or elsewhere in the system 10). The time ranges (e.g. elapsed time, maximum allowable time out of storage) may be displayed on the handheld 22 with a visual indicator indicating how much time is left to return the unit to the blood bank. Such visual indicators may be different colors, different symbols, etc. Additionally, if a particular unit has to be used or returned to the blood bank imminently (i.e. before the sample is deemed too long out of storage and must be destroyed), the handheld device 22 may emit an audible sound/visual warning that alerts the user to that fact.

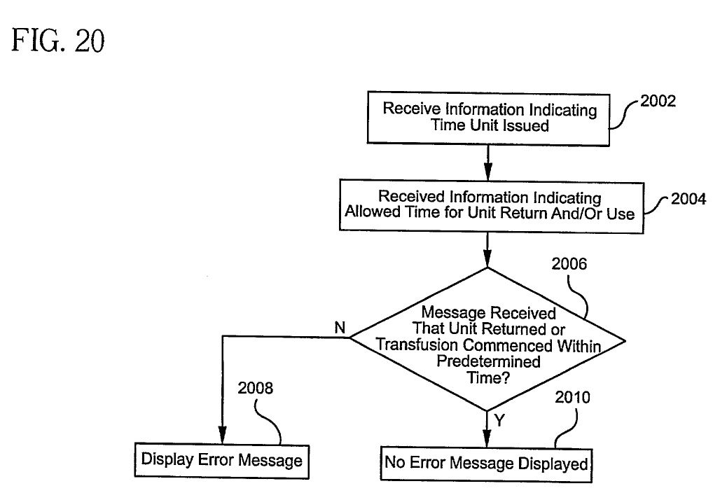

FIG. 20 illustrates the process for monitoring the time for allowing a unit to be returned to a blood bank or used—before it must be disposed. At step 2002, system 10 receives information indicating the time that a unit was released from a blood bank. In addition, database 44 stores information that is accessible to (and may be stored by) handheld device 22 that indicates the amount of time before the unit must be used or returned to the blood bank—in order for the unit to be acceptable for transfusion. At step 2006, handheld device 22 monitors for whether the time period of such use or return has been met and whether, within that time period, data has been received indicating the unit's use or return. If the unit is not used within the predetermined timeframe, then an error message is displayed by handheld device 22 (step 2008). If, however, the unit is used within the predetermined timeframe, then no error message is displayed (step 2010).

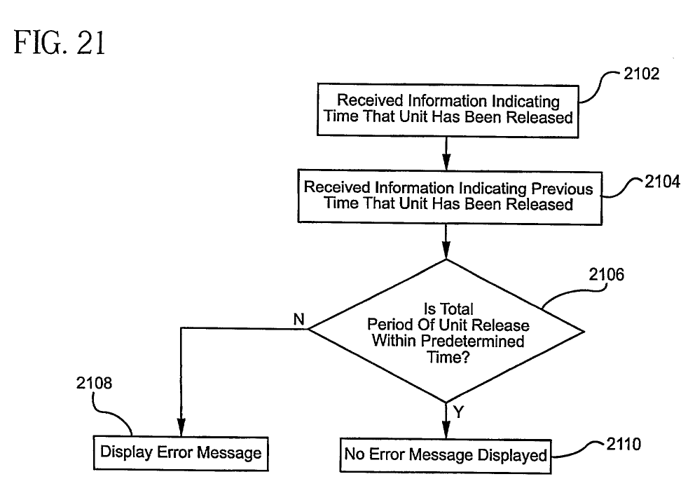

In addition, to tracking whether the a unit is used or returned to the blood bank within a predetermined time from when it is released, system 10 can monitor for the total time that a unit has been released from the blood bank—even when the unit has been released from the blood bank multiple times. This may occur if, for example, a unit is released two or more times and the total release time is of relevance to determining the integrity of the unit. Thus, at step 2102 of FIG. 21, handheld device 22 receives information indicating the time that a unit has been released. At step 2104, handheld device 22 receives information about the duration of time in which the unit has been previously released. Next, at step 2106, handheld device determines whether the total release time exceeds a predetermined amount of time for the unit. If such predetermined time period is exceeded, then an error message is displayed by handheld 22 (step 2108). If, however, such predetermined time period is not exceeded, then no error message is displayed (step 2110).

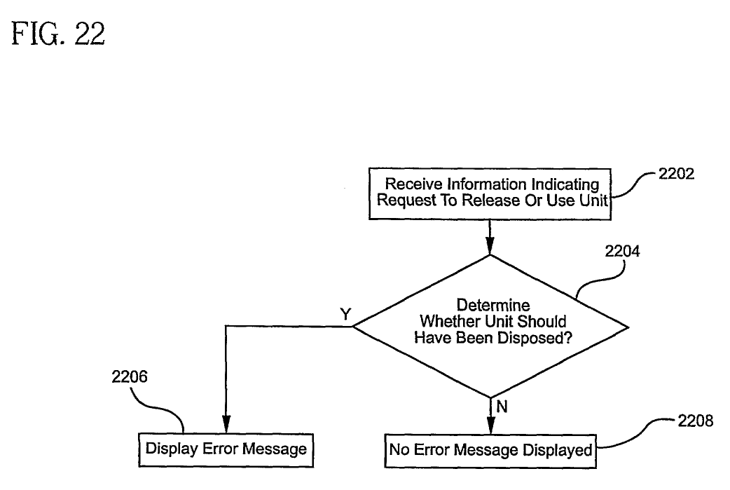

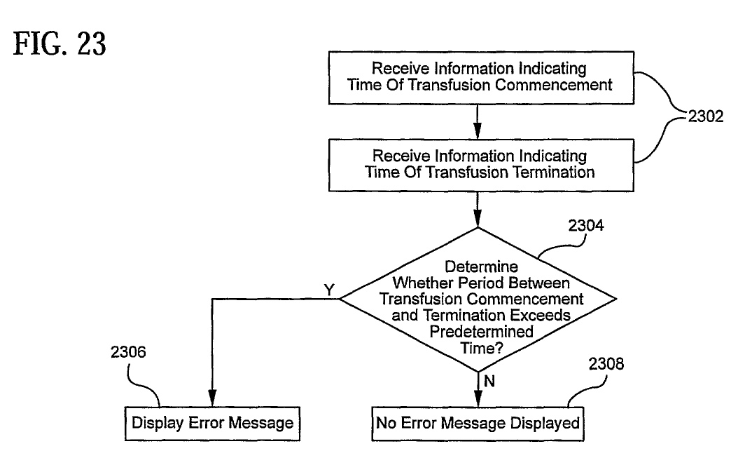

As described above, there are instances when a unit should be disposed—for example, when a unit's expiration date passes or a unit is released beyond a predetermined time. FIG. 22 illustrates the process for generating an error message when a unit that is intended to be disposed is requested for a transfusion. At step 2202, handheld unit 22 receives a request for a unit to be released. In accordance with an embodiment of the invention, handheld 22 determines the requested unit is one that should have been (or already has been) disposed (step 2204). If the unit has been or should have been disposed, the handheld device 22 displays and error message (step 2206). If, however, the unit has not been or should not have been disposed, no error message is displayed (step 2208).

In one embodiment, the system is configured to obtain information to provide an “audit trail” for each unit. In this embodiment, information is entered into the system about the unit, when in leaves the blood bank, when it reaches the patient location and, if it is returned, when it is returned to the blood bank. For example, when a unit is returned to the blood bank, the user may have to scan their user ID, the unit ID and enter other information concerning the return of the unit. Examples of such other information include: why it was returned; time it was returned; patient it came from; unit number; user's electronic signature; and time and date of return. Other examples of information that the system might be configured to obtain in order to provide a complete audit trail for the unit are readily apparent to one skilled in the art.

In addition to providing an audit trail for the units, the system can be used to monitor the transfusion process and assist the medical professional in administering the transfusion. For example, some transfusion specifications set guidelines for how long blood may be transfused into a patient. For instance, some guidelines set a four hour window for transfusing blood. Because the handheld device 22 can be taken to the location where the patient is to be transfused, the handheld device 22 can be used to communicate the time at which the transfusion starts to the rest of system 10. In one embodiment, the user activates the start transfusion icon on the handheld device 22. This records the transfusion start time for the specific unit (step 2302). The elapsed time for the transfusion may be displayed on the handheld device 22 or another display screen such as on the workstation 28. The system processor has stored therein data for the maximum transfusion time and begins monitoring the time elapsed against the maximum permissible time. The transfusion termination time may also be received by the handheld device (step 2302) and the time between transfusion commencement and termination may thereby be compared with the maximum time allowed for transfusion (step 2304). If the transfusion is completed within the predetermined maximum time, then no error message is displayed (step 2308). If, however, the transfusion is not completed within the predetermined maximum time, then an error message is displayed by handheld device 22 (step 2308). The system may cause the handheld device 22 to provide a visual indication or emit an audible sound when the maximum permissible transfusion time has elapsed.

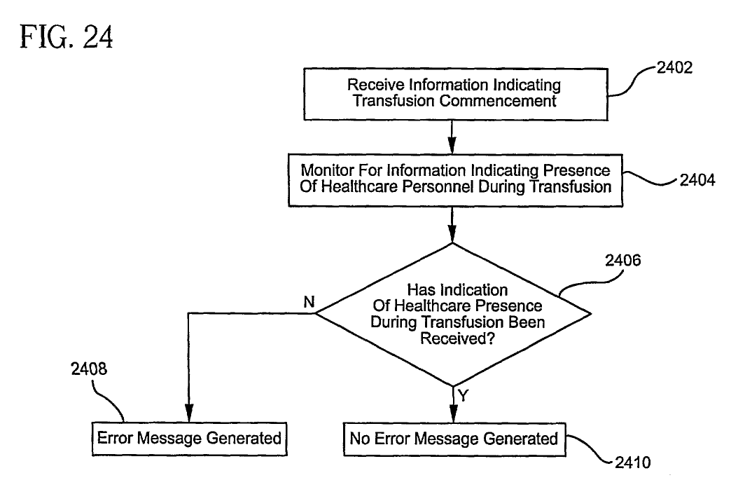

In some instances, the user is required to stay in the room for a period of time after the transfusion has started. In one embodiment of the present invention, the handheld device 22 provides a message informing the user how long the user must remain in the room. In other embodiments, the handheld is equipped with a position detector such as global positioning system (GPS). In this embodiment, when the user exits the room with the handheld before the time for monitored transfusion has expired, the handheld device emits an audible or visual signal to alert the user that he/she should go back to the room until the time for monitored transfusion has elapsed. In another embodiment, depicted in the flowchart of FIG. 24, the handheld device receives information indicating that a transfusion has commenced (step 2402) and monitors for information indicating the presence of a healthcare personnel during transfusion (step 2404). The information may be generated by the scan of the patient's wristband every five or ten minutes. At step 2406, handheld device 22 monitors whether such indication is received. If the indication is not received, an error message is generated by handheld device 22 (step 2408). If, however, the indication is not received, no error message is generated (step 2410).

In accordance with an embodiment of the invention, after scanning the unit's information, the handheld device 22 prompts the user to scan the patient's identification number which may be situated on the patient's wristband and associated Hollister number (if applicable). Once the patient information is received, BTS 20 compares the patient data with the unit data to determine if there is a match between the data such that the transfusion process may continue.

The user may also be prompted to confirm the blood type of the unit. This is indicated by prompt 672 in FIG. 13. There are four basic blood types: A, B, AB, and O. Type AB blood type is known as a universal acceptor because it can receive all four blood types without causing complications (as long as the Rh factors match). Type O blood is known as the universal donor because it can be given to patients of any blood type. If, for example, a person with type A blood is given type B blood, complications, including fatality may occur. As such, the handheld device 22 or system may be configured to run a simple check between the patient's blood type and the blood type of the unit. If they do not match or the two types would conflict and cause complications or fatality, the handheld device may issue a visual and/or audible warning. The unit may also be configured to match the Rh factor of the patient and the unit. The blood types and Rh factors may be indicated in different colors on the display screen.

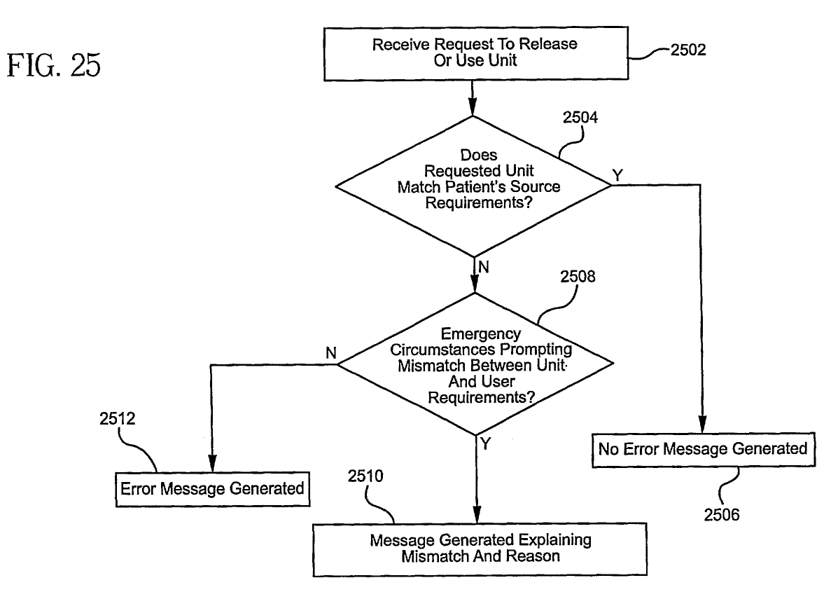

Turning to FIG. 25, a process is illustrated to ensure that a patient is administered the appropriate source of unit. There are typically three sources for blood used in blood transfusions, autologous (from the patient), directed (from a specific donor for the patient) or allogenic (from a random donor). The order of preference for blood use is autologous, directed, and then allogenic. For instance if the unit scanned is allogenic blood and the system has the patient linked to directed blood, the handheld device may alert the user to this fact. However, there may be an emergency situation where there is no time to get the directed blood. As such, the user may override the handheld device alert and may enter a reason for not using the directed blood into the handheld device. When the user overrides the handheld device 22, a dropdown menu is provided so the user can select the reason for the override. Alternatively, the user may be prompted to enter the reason for the override using the keyboard provided by the handheld device. This may allow the user to keep an accurate record of what has transpired in real time for later use. Thus, at step 2502, handheld device 22 receives a request to use a certain unit. Handheld device 22 determines or receives information to enable a determination as to whether the requested unit matches the source information associated with the patient (step 2504). If the request matches the patient's source requirements (i.e., autologous, directed or allogenic), no error message is displayed (step 2506) and the transfusion process may proceed. If, however, the request does not match the patient's source requirements, the handheld device 22 prompts the user to send information to handheld device 22 indicating whether an emergency circumstance exists prompting a mismatch between the unit and the patient's source requirements (step 2508). If there is indeed an emergency, a message is generated for storage by system 10 explaining the emergency status contributing to the mismatch (step 2510). If however, no emergency existed, an error message is generated (step 2512).

In another embodiment of the invention, system 10 may also monitor the American Association of Blood Banks (AABB) requirement that the unit be maintained in a specific temperature range. The handheld device 22 may prompt the user to check the temperature of the unit. If the temperature of the unit exceeds the temperature range, the handheld device 22 directs users to discard that particular unit.

Various specific features of system 10 have been described. The typical sequential order of the transfusion process may be summarized in four phases: 1) pre-transfusion; 2) issue; 3) unit receipt; and 4) unit return phases of the transfusion process. Initially, the user logs into system 10 (as described above) and selects the transfusion module. After which, the user retrieves the patient info by scanning the patient's wristband using, for example, scanner of handheld device 22. The handheld device 10 displays the patient information and the user is directed to verify the doctor's blood bank order, patient's consent signature, type of unit or product requested (e.g. whole blood or packed cells) and the number of units requested. Then the user indicates that the order is complete. The order is transmitted from the handheld device 22 to the blood bank that is networked with the system 10. If the handheld device 22 is configured to communicate wirelessly with the system, the order can be sent immediately through the wireless connection. Otherwise, the order is transmitted when the handheld device is placed back in the cradle and synchronized with the system.

The blood bank also enters the issuing information such as unit number, date and time issued, etc. to the system 10. The unit is then transported by messenger, pneumatic transport system or any other suitable means to a specified location. As previously described, the system 10 may have a webpage that displays the status of the ordered unit (i.e., the time released from the blood bank, an indication that the unit is in transit, etc.). The webpage may also display the time window in which the unit must be used or returned before the unit must be discarded. The time window can be displayed in color coded manner, so that the user can receive a quick visual reference for the amount of time remaining (e.g. blue for one or more hours, red for less than one hour, etc.). The information displayed from the webpage can be displayed on the handheld device 22.

When the unit is received, the user once again logs into the system 10 and scans the unit. The user then selects the “unit received” option and the system, in response, records the date and time the unit was received. The system then determines whether or not the patient is ready to be transfused. If the patient is not ready but a time window for safe return of the unit set by the hospital (and entered into the system 10) has not elapsed (for example, 30 minutes), then the user is prompted to return the units to the blood bank. If the unit is to be returned, the unit is scanned, and the return option is selected on the handheld device 22. The handheld device 22 displays the patient information and indicates to the user that it is permissible to return the unit to the blood bank. In response, the system 10 changes the unit status and date and time of return and updates the information stored by database 44.

If the unit is not returned within the previously set window (and the unit has not been transfused into the patient), then the handheld device 22 displays the patient information and indicates that it is not permissible to return the unit. Once again, the system changes the status of the unit and updates the information stored in database 22 relative to that unit to remove it from the stored inventory of available units.

Once a transfusion is ready to proceed, the user scans the patient's wristband and the unit's identification information, so that the system can determine if there is a match between the patient and the unit in the system. If the patient's identification information does not match the unit number, then the transfusion process is stopped by alerting the user, the blood bank is notified and the unit is returned. If the patient's information does match the unit information, the user is required to verify particular secondary information such as patient first and last name, patient MR number, patient blood group and type, donor blood group and type, donor expiration date, special requirements, product type and compatibility, and the like. This data verification ensures that there is a match, and thereby adds reliability to the system. After the data is verified, the system 10 indicates to the user that the transfusion may commence. The user then begins the transfusion sequence as previously described.

With the termination sequence of the transfusion process, in one instance, the unit number is scanned and the reason for terminating the transfusion is entered by the user through the previously described drop down menu displayed on the handheld device 22 (e.g. the transfusion was completed, the maximum allowable transfusion time elapsed, transfusion reaction, etc.). The system may then print a label for the patient's medical chart. The label may include information specific to the particular transfusion.

The system 10 may implement additional steps in the transfusion sequence for receiving additional information about the unit. This ensures that the unit is indeed the correct unit for the patient identified in the system 10 to be transfused with the unit. Thus, after the system 10 has determined that there is a match between the patient's primary identification and the scanned unit, a secondary identifier on the unit is scanned or manually entered by the user through the handheld device 22. The secondary identifier is, for example, a Hollister number given by the blood bank. Next a determination is made as to whether the Hollister number for the unit matches the patient. If it does, the transfusion proceeds. If the Hollister number on the unit does not match the Hollister number for the patient, the transfusion process is stopped.

Further steps may be implemented in the transfusion sequence for receiving additional information about the unit's association with the patient to be transfused. After the system 10 determines that the patient's primary identifier matches the unit information, a secondary identifier on the unit is scanned or manually entered by the user. The secondary identifier may be an accession number given by the blood bank. Next, the system 10 determines whether the accession number for the unit matches the scanned patient information. If there is a match, then the system 10 indicates that the transfusion can proceed, and manages that transfusion as previously described. If the system determines that the accession number on the unit does not match the scanned patient information, the transfusion process is stopped.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

For example, while the system 10 and the associated processes were described in connection with transfusions many aspects of the system 10 and processes may be implemented in other healthcare systems that require the monitoring of time-sensitive and/or patient-specific medical procedures.

In addition, while many of the processes were processed by handheld 22 which is synchronized with system 10, wireless handheld device 32 could also effectuate the processes in real-time or close to real-time. In addition, much of the processing may be performed by a central server, such as server 20.

What is claimed is:

1. A system for monitoring a medical treatment unit, comprising:
a storage device configured to receive data indicating an amount of time that the medical treatment unit is released;
a processor configured to compare whether the amount of time exceeds a predetermined time threshold; and
a display unit configured to generate a message when the amount of time exceeds the predetermined time threshold.

2. The system of claim 1, wherein when the amount of time exceeds the predetermined time threshold, the display message indicates that the medical treatment unit is to be returned to a unit originator.

3. The system of claim 1, wherein when the amount of time is exceeds, the display message indicates that the medical treatment unit is to be disposed.

4. The system of claim 1 wherein at least a portion of the system is in a handheld device.

5. A system for monitoring a medical treatment unit, comprising:
a storage device configured to:
receive data indicating a first amount of time, the first amount of time measures a duration that the medical treatment unit is released; and
receive data indicating a second amount of time, the second amount of time measures a duration that the medical treatment unit has been previously released;
a processor configured to:
total the first amount of time and the second amount of time; and
compare whether the totaled amount of time exceeds a predetermined threshold; and
a display unit configured to generate a message when the totaled amount of time exceeds the predetermined time threshold.

6. The system of claim 5, wherein when the totaled amount of time exceeds the predetermined time threshold, the display message indicates that the medical treatment unit is to be disposed.

7. The system of claim 5 at least a portion of the system is in a handheld device.

8. The system of claim 7 wherein at least the display unit is the handheld device.

9. A system for monitoring a medical treatment unit, comprising:
a storage device configured to access medical treatment unit data upon a request for the medical treatment unit;
a processor configured to determine, based upon the medical treatment unit data, whether the medical treatment unit has been designated for disposal or has been previously requested; and
a display unit for displaying a message when the medical treatment unit has been designated for disposal or as previously requested in response to the determination by the processor.

10. A system for monitoring a medical treatment, comprising:
a storage device configured to receive a medical treatment start time and medical treatment end time;
a processor configured to determine whether, based upon the medical treatment start time and the medical treatment end time, the medical treatment has exceeded a predetermined time threshold; and
a display configured to generate a message when the medical treatment has exceeded the predetermined time threshold.

11. A system for monitoring a medical treatment, comprising:
a storage device configured to receive the location of the medical treatment and for receiving a start and end time of the medical treatment;
a sensor configured to detect whether a device associated with the administrator of the medical treatment is in the location of the medical treatment during at least one of the start and the end time; and
a display configured to generate a message when the device associated with the administrator of the medical treatment is not in the location of the medical treatment during the start and the end time.

12. The system of claim 11 wherein at least a portion of the system is a handheld device.

13. The system of claim 12 wherein at least the sensor and the display are in the handheld device.

14. A system for monitoring a medical treatment provided to a patient by an administrator, comprising:
a storage device configured to receive a start time and an end time of the medical treatment;
a processor configured to generate a request that the administrator scan identification information associated with a label of the patient between the start time and the end time;
a display configured to generate a message if no identification information associated with a label of the patient between the start time and the end time is received.

15. The system of claim 14 wherein at least a portion of the system is in a handheld device.

16. The system of claim 15 wherein the handheld device further comprises a scanner.

17. A system for monitoring a medical treatment unit to be provided to a patient by an administrator, comprising:
a storage device configured to:
receive patient-specific medical treatment unit source information; and
receive information pertaining to a source of the medical treatment unit to be provided to the patient;
a processor configured to determine whether the patient-specific medical treatment unit source information matches the source of the medical treatment unit to be provided to the patient; and
a display configured to generate a message when the patient-specific medical treatment unit source information does not match the source of the medical treatment unit to be provided to the patient.

18. The system of claim 17 wherein the medical treatment unit is blood and the patient specific medical unit source information indicates if the blood is one of autologous, directed or allogenic.

19. The system of claim 18 wherein the medical treatment unit is blood and the information pertaining to a source indicates if the blood is one of autologous, directed or allogenic.

20. The system of claim 19, wherein the processor requests emergency information, when the patient-specific medical treatment unit source information does not match the source of the medical treatment unit to be provided to the patient.

21. A system for monitoring a medical treatment unit, comprising:
a processor that sends a prompt signal to measure the temperature of the medical treatment unit and compares the measured temperature to a predetermined temperature received by the processor;
an input device adapted to receive the measured temperature and communicate the measured temperature to the processor;
a storage device that stores the predetermined temperature retrieved by the processor and compared with the measured temperature; and
a user interface unit configured to communicate the prompt signal and the results of the comparison to the user.

22. The system of claim 21 wherein the user interface unit indicates that the medical treatment unit is to be disposed when the measured temperature exceeds the predetermined temperature.

23. The system of claim 21 wherein at least a portion of the system is in the handheld device.

24. The system of claim 23 wherein at least the user interface unit is in the handheld device.

25. The system of claim 24 wherein the user interface unit is a display unit.

26. A system for administering medical treatment, the system comprising:
a server having information pertaining to at least one patient and at least one medical treatment unit;
a processor for using the patient information and the medical treatment unit information to administer medical treatment;
a communication link between the server and the processor;
a handheld device operable to receive and output patient information and medical unit information, the handheld device further comprising:
a transceiver operable to communicate with the server to transmit and receive the patient information and the medical unit information; and
a display for displaying at least a portion of the received patient information and the received medical unit information.

27. A method for monitoring a medical treatment unit, comprising:
receiving data indicating an amount of time that the medical treatment unit is released from a storage location;
comparing whether the amount of time exceeds a predetermined time threshold; and
generating a message when the amount of time exceeds the predetermined time threshold.

28. The method of claim 27, wherein, when the amount of time exceeds the predetermined time threshold, the display message indicates that the medical treatment unit is to be returned to a storage location.

29. The method of claim 27, wherein when the amount of time exceeds the predetermined time threshold, the display message indicates that the medical treatment unit is to be disposed.

30. A method for monitoring a medical treatment unit, comprising:
receiving data indicating a first amount of time, the first amount of time being a measure of a duration that the medical treatment unit is released;
receiving data indicating a second amount of time, the second amount of time measures a duration that the medical treatment unit has been previously released;
totaling the first amount of time and the second amount of time;
comparing whether the totaled amount of time exceeds a predetermined threshold; and
generating a message when the totaled amount of time exceeds the predetermined time threshold.

31. The method of claim 30, wherein when the amount of time is exceeds, the display message indicates that the medical treatment unit is to be disposed.

32. A method for monitoring a medical treatment unit, comprising:
accessing medical treatment unit data upon a request for the medical treatment unit;
determining, based upon the medical treatment unit data, whether the medical treatment unit has been designated for disposal or has been previously requested; and
generating a message when the medical treatment unit has been designated for disposal or as previously requested.

33. A method for monitoring a medical treatment, comprising:
receiving a medical treatment start time and medical treatment end time;
determining whether, based upon the medical treatment start time and the medical treatment end time, the medical treatment has exceeded a predetermined time threshold; and
generating a message when the medical treatment has exceeded the predetermined time threshold.

34. A method for monitoring a medical treatment, comprising:
receiving the location of the medical treatment;
receiving a start and end time of the medical treatment;
determining whether a device associated with the administrator of the medical treatment is in the location of the medical treatment during the start and the end time; and
generating a message when the device associated with the administrator of the medical treatment is not in the location of the medical treatment during the start and the end time.

35. A method for monitoring a medical treatment provided to a patient by an administrator, comprising:
receiving a start time and an end time of the medical treatment;
generating a request that the administrator scan identification information associated with a label of the patient between the start time and the end time;
generating a message if no identification information associated with a label of the patient between the start time and the end time is received.

36. A method for monitoring a medical treatment unit to be provided to a patient by an administrator, comprising:
receiving patient-specific medical treatment unit source information;
receiving information pertaining to a source of the medical treatment unit to be provided to the patient;
determining whether the patient-specific medical treatment unit source information matches the source of the medical treatment unit to be provided to the patient; and
generating a message when the patient-specific medical treatment unit source information does not match the source of the medical treatment unit to be provided to the patient.

37. The system of claim 36 wherein the medical treatment unit is blood and the patient specific medical unit source information indicates if the blood is one of autologous, directed or allogenic.

38. The system of claim 37 wherein the information pertaining to a source indicates if the blood is one of autologous, directed or allogenic.

39. The method of claim 38, wherein the processor requests emergency information, when the patient-specific medical treatment unit source information does not match the source of the medical treatment unit to be provided to the patient.

40. A method for monitoring a medical treatment unit, comprising:
sending a prompt signal to measure the temperature of the medical treatment unit;
receiving the measured temperature;
comparing the measured temperature to a predetermined temperature; and
when the measured temperature exceeds the predetermined temperature, sending a signal indicating that the measured temperature exceeds the predetermined temperature to a user.

41. A computer-based method for monitoring patient treatment over a network having at least one server and a processor in communication therewith, the method comprising the steps of:
inputting at least one of first patient information and first medical unit information into a system;
inputting at least one of second patient information and second medical unit information into a handheld device which communicates with the system via a network communication link; and
comparing the first information with the second information; and
outputting to the handheld device an instruction for administering the medical unit to the patient based on the comparison.
